(19) United States
(12) Patent Application Publication
(10) Pub. No.: US 2023/0330361 A1
(43) Pub. Date: Oct. 19, 2023

(54) CAPSULE INHALER, METHOD FOR MONITORING INTAKE OF A SUBSTANCE, COMPUTER PROGRAM AND COMPUTER PROGRAM PRODUCT

(71) Applicant: PULINNO SP. Z O. O., Bydgoszcz (PL)

(72) Inventors: Pawel ROSZCZYK, Warszawa (PL); Tomasz SOSNOWSKI, Warszawa (PL); Arkadiusz MOSKAL, Cegielnia (PL); Emil KLUCZ, Znin (PL); Mscislaw SRUTEK, Bydgoszcz (PL); Slawomir BUJNOWSKI, Koronowo (PL); Mateusz WIRWICKI, Bydgoszcz (PL); Waldemar WISNIEWSKI, Bydgoszcz (PL); Krzysztof KLUCZ, Ciele (PL)

(21) Appl. No.: 18/042,814

(22) PCT Filed: Aug. 23, 2021
(86) PCT No.: PCT/IB2021/057721

§ 371 Date: Feb. 24, 2023

(30) Foreign Application Priority Data
Aug. 24, 2020 (PL) P.435072

(51) Int. Cl. A61M 15/00

(52) U.S. Cl. CPC A61M 15/0025; A61M 15/0035; A61M 15/0041; A61M 2016/0018; A61M 2202/064; A61M 2205/3317; A61M 2205/581

(57) ABSTRACT

The invention provides a capsule inhaler for administering a single dose of dry powder, comprising a body and a mouthpiece, and a cover, coupled with the body. Additionally, the inhaler comprises a capsule receiving element and press buttons with a capsule piercing mechanism as well as an electronic board comprising a microprocessor, a magnetic field sensor and a light indicator, positioned close to the press button that comprises a piercing element and a magnet. Moreover, the invention provides a method for monitoring intake of a substance by means of a capsule inhaler, comprising steps of controlling intake of a substance and providing feedback to the user during intake of the substance. The invention also provides a computer program and a computer program product capable to execute steps of the method.

The invention provides a smart capsule inhaler of a variable resistance parameter, ensuring pulmonary deposition suitable for specific substance formulation, for example a drug in a dry powder form enclosed in a cellulose or gelatine capsule, a method for monitoring proper intake of the substance with the use of such smart inhaler, a computer program for monitoring proper intake of the substance with the use of such method and a related computer program product.

There are known devices for ensuring proper pulmonary deposition, both in a form of caps or accessories for inhalers, as well as smart inhalers as such having a structure that ensures proper administering of a full drug dose, that guide the inhaler user throughout a proper inhalation process.

Document GB 2542910 A discloses a device and a method for ensuring proper drug intake with the use of a capsule inhaler that uses a dedicated band for the user. The band comprises a power source, a sensor to detect proximity of the inhaler, a controller, a memory, a time measurement unit and a communication unit. The band, by means of the detecting sensor, checks periodically for proximity of the inhaler and this is possible due to a marker provided on the inhaler, and when the inhaler is detected, it is activated to capture and record data transmitted from the inhaler, and then to send the data to a home memory and to transmit instructions, via the home memory, for proper executing of the inhalation process. One of the most important aspects of the invention is provision of low energy consumption by the inhaler and the band.

Similar solution is disclosed in US 10524726 B2 which describes a system for ensuring proper drug intake by a patient, comprising a band wearable by the user for collecting data concerning the drug intake. The band is operable to send data collected from the user to a network. The solution disclosed in US 10524726 B2 has sensors positioned on the band itself and not on the drug administering device, and it collects information directly from the user’s body.

From CN 105163784 B a device, a system and a method are known for monitoring a device for intake of drugs for respiratory tract, tracking the frequency and effectiveness of the drug intake. The system disclosed is configured so that it turns on upon opening of the device cover and memorizes, by means of a processor, the data provided from its sensors, the sensors collecting information on the drug intake when the device is used by the patient.

Devices known from the prior art offer methods to ensure that the drug has been administered to the user of an inhaler as well as methods to transmit feedback based on data collected during intake of the drug by the user, but quite often they do not transfer such information to the user in real time. Apart from that, for complete monitoring they require additional devices such as bands, or they relate to caps and not to inhalers as such, and often they do not transmit feedback to the user directly and thus they do not make it possible to correct the inhalation process as it proceeds. The solutions of the prior art do not provide automatic transmittal of complex instructions to the user and they require home memory. There is a lack of solutions to ensure comprehensive monitoring of drug intake, dedicated for capsule inhalers, including inter alia verification of correctness of inhalation process and transmitting suitable feedback to the user in real time.

The capsule inhaler for administering a single dry powder dose from a capsule, comprising a body, a mouthpiece with a sieve and a mouthpiece base and a cover with an opening, coupled with the body, and a capsule receiving element and at least one press button with a capsule piercing mechanism, as well as at least one electronic board, is characterized in that the electronic board comprises a microprocessor, at least one position sensor, and it comprises at least one magnetic field sensor and at least one light indicator, positioned close to at least one press button comprising at least one piercing mechanism and at least one magnet. Additionally, the electronic board is coupled, via a coupling element, with the base of the mouthpiece and it comprises at least one pressure sensor.

Preferably, the piercing mechanism comprises at least one spring and at least one piercing element.

Preferably, the magnetic field sensor is a Hall-effect device.

Preferably, the light indicator is a diode.

Preferably, the position sensor is an accelerometer.

Preferably, the electronic board comprises a switch which, via a coupling element, is coupled with the base, and preferably the coupling element is a pusher.

Preferably, on the electronic board, coupled with a microprocessor, an antenna is positioned, and preferably the antenna is a Bluetooth antenna.

Preferably, on the electronic board, coupled with a microprocessor, a connector is positioned, and preferably the connector is a USB-C-type connector.

Preferably, on the electronic board, coupled with a microprocessor, an on/off switch is positioned.

Preferably, on the electronic board, coupled with a microprocessor, a resetting element is positioned, where preferably the resetting element is a resetting switch.

Preferably, on the electronic board, coupled with a microprocessor, a real time clock is positioned.

Preferably, on the electronic board, an audio indicator is positioned.

Preferably, in a frame in proximity of the electronic board a battery is positioned.

Preferably, the sieve comprises a mesh with rectangular openings of a width comprised between 0.94 and 1 mm and a length comprised between 0.97 and 1.03 mm and a spacing between the openings between 0.47 and 0.53 mm.

Preferably, the sieve comprises a mesh with rectangular openings of a width comprised between 0.68 and 0.72 mm and a length comprised between 1.07 and 1.13 mm and a spacing between the openings between 1.37 and 1.43 mm.

Preferably, the sieve comprises a mesh with rectangular openings of a width comprised between 1.08 and 1.14 and a length comprised between 1.07 and 1.13 mm and a spacing between the openings being between 0.90 and 0.96 mm.

A method for monitoring intake of a substance with the use of a capsule inhaler is characterized in that the method comprises executing, by a processing unit, steps of:
periodically activating of a processing unit;
verifying whether Bluetooth is active;
verifying the status of the process in the inhaler memory;
verifying opening of the mouthpiece base by monitoring a coupling element;
activating and reading out the status of sensors;
executing inhalation step comprising:
   verifying closing of a mouthpiece base by monitoring a coupling element;
   verifying pressing of press buttons by checking whether signal from magnetic field sensors exceeds a predetermined threshold value;
   verifying, by a position sensor, the position of the inhaler;
   checking the status of a pressure sensor that monitors breathing-in,
   verifying the value of the breath-in force and breath-in duration time;
   ending the inhalation step;
verifying ending of the inhalation step.

Preferably, verification of the status of the process in the inhaler memory comprises verification of the inhaler memory setting and, upon positive inhaler memory verification result, recording, in the inhaler memory, data from the sensors, verification of the duration time count-down of the inhaler memory setting and zeroing the inhaler memory cell setting, and then passing to the step of verification of opening of the base.

Preferably, verification of the status of the process in the inhaler memory comprises verification of the inhaler memory setting and, upon negative inhaler memory setting verification result, passing to the step of verification of opening of the mouthpiece base.

Preferably, verification of the status of the process in the inhaler memory comprises verification of the inhaler memory setting and, upon positive inhaler memory setting verification result, recording, in the inhaler memory, data from the sensors and further verification of the inhaler memory duration time count-down setting, and with negative inhaler memory duration time count-down setting verification result, returning to the step of verification of the inhaler memory setting.

Preferably, with negative mouthpiece base opening verification result, the method returns to the step of periodically activating of the processing unit.

Preferably, with positive breath-in force value verification result and breath-in duration time verification result, additionally opening of the mouthpiece base is verified.

Preferably, in the step of verification of ending of the inhalation step at least one of the following operations is executed: checking whether a maximum time inhalation elapsed, verifying the status of the inhalation step.

Preferably, in the step of verification of ending of the inhalation step, upon occurrence of at least one of the following events: elapse of a maximum inhalation duration time, ending of the inhalation step, the step of periodically activating of the processing unit is executed.

Preferably, in the step of verification of ending of the inhalation step, upon occurrence at least one of the following events: negative maximum inhalation time elapse verification result, ending of the inhalation step, the step of putting the sensors into a standby mode is executed.

Preferably, following the step of setting the sensors into a standby mode, the method returns to the step of activating and reading out the status of the sensors.

Preferably, upon each positive verification result in the inhalation step, the step of signalling by means of a light indicator and/or audio indicator is executed.

Preferably, upon each negative verification result of closing of the base, verification of the position of the inhaler and with a negative result of the check of the status of the pressure sensor, a step of verification whether the duration time exceeds a predetermined value is executed.

Preferably, upon each negative result of verification of pressing of the press buttons and verification of the breath-in force and breath-in duration time, the step of signalling by means of the light indicator and/or audio indicator is executed.

Preferably, before the step of verification of closing of the mouthpiece base, a step of signalling by means of the light indicator is executed.

Preferably, in the step of activating and reading out the status of the sensors, the status of at least one of them is read out: a switch, magnetic field sensor, pressure sensor, position sensor, real time clock.

Preferably, the processing unit is a microprocessor.

Preferably, the position sensor is an accelerometer.

Preferably, maximum inhalation time is between 16 and 180 seconds.

Preferably, the step of verification of the inhaler position is completed when the inclination angle is within a range of 10°-20° +/-2° to the horizon.

Preferably, upon positive Bluetooth activity verification result, the method is executed by an application on the user’s device.

Preferably, the user’s device is a smartphone.

A computer program for monitoring intake of a substance comprises instructions for execution of a method for monitoring intake of a substance.

A computer program product comprising a computer readable code, to execute steps of a method for monitoring intake of a substance.

Inhaler according to the invention generates an aerosol from a solid form of a drug comprising an active substance in a form embedded in a lactose carrier, under the flow of air caused during the user (patient) breath-in. This process requires overcoming internal resistance of the inhaler and aerodynamic resistance. Effectiveness of inhalation depends on forming of a breath-in flow ensuring proper disaggregation of the drug that determines generation of a micromolecular fraction. In turn, proper construction of the inhaler determines the rate and nature of pulmonary deposition to provide a high therapeutic effectiveness of the inhaled drugs. The inhaler is a one-dose device that requires a capsule with the drug to be inserted to the inhaler chamber and pierced by a needle mechanism.

The principal functionalities of the inhaler according to the invention include:
transmitting information concerning opening, closing of the inhaler;
measurement of pressure drop caused by the air flow in the inhaler;
possibility to determine the inhaler position in a XYZ coordinate system;
possibility to monitor motion of the press buttons that pierce the capsule with the drug;
possibility to establish a Bluetooth connection with a mobile phone;
possibility to record data from several measurements in the inhaler memory;
light and audio communication with the user;
dedicated application to enable collecting data from the device, with a function of training of the user for proper use of the inhaler;
application to indicate a high probability of intake of the whole therapeutic dose of a specific drug.

Monitoring method according to the invention ensures proper intake of a substance dose by the user of the inhaler according to the invention.

The principal functionalities of the method according to the invention include identical functionalities as the ones of the inhaler and a possibility to guide the user throughout the inhalation process, while presenting real time instructions for proper inhalation and information on errors in the inhalation process.

The object of the invention is shown in the drawing in which:

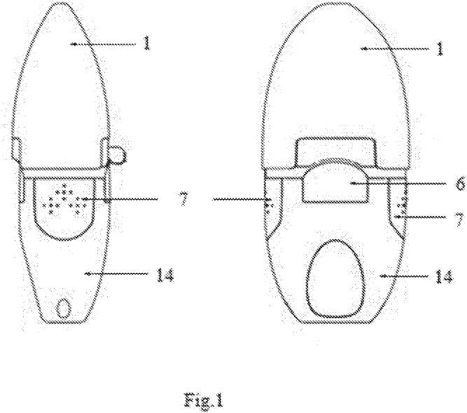

FIG. 1 shows an inhaler according to the invention, in a closed configuration with a cover, in a front and side views of the inhaler;

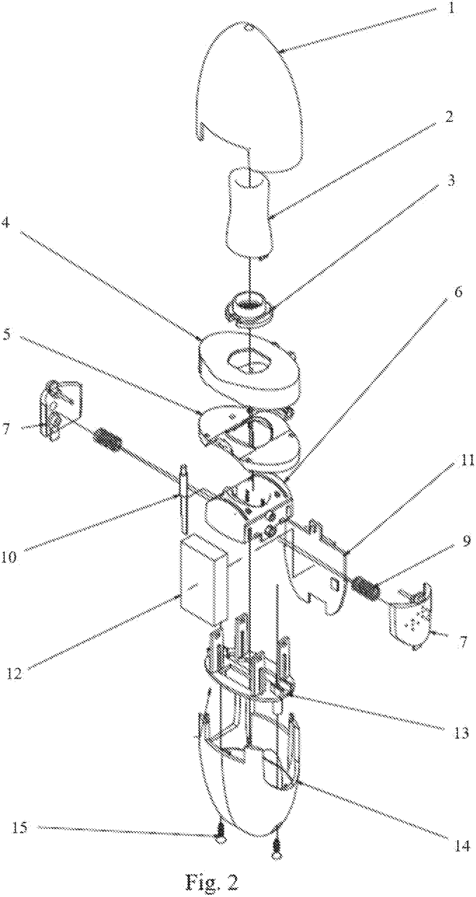

FIG. 2 shows an exploded view of an inhaler according to the invention;

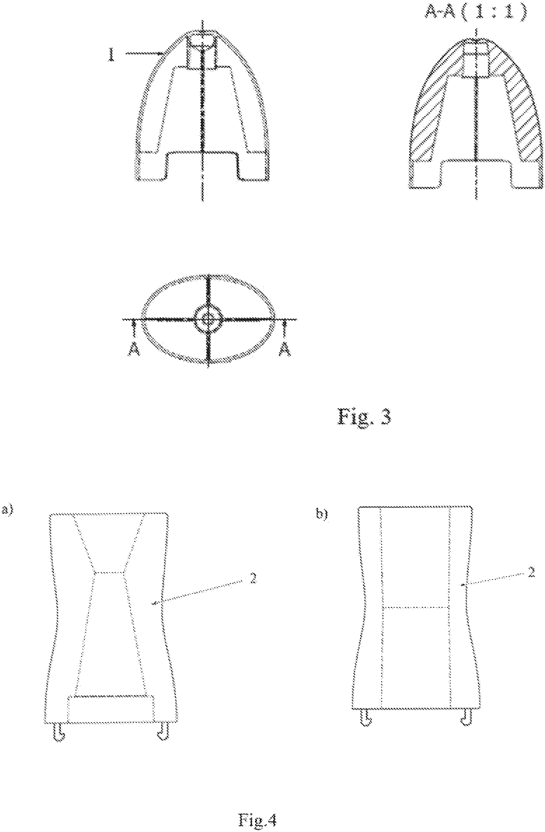

FIG. 3 shows a housing of an inhaler according to the invention, in cross-sectional views;

FIG. 4a shows a mouthpiece of an inhaler according to the invention with a dashed line indicating a channel within the mouthpiece and with visible catches;

FIG. 4b shows a mouthpiece of an inhaler according to the invention with a dashed line indicating a channel, other than the one in FIG. 4b, inside the mouthpiece and with visible catches;

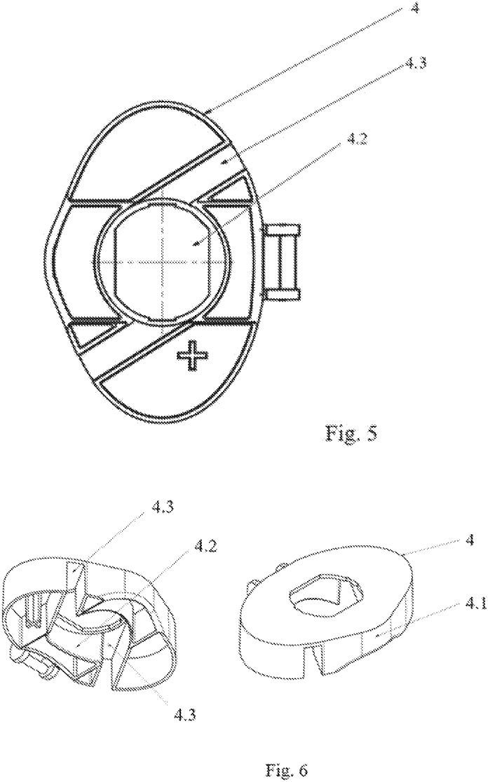

FIG. 5 shows a base of a mouthpiece of an inhaler according to the invention, in a bottom view with a visible inlet channel and a rotatable chamber;

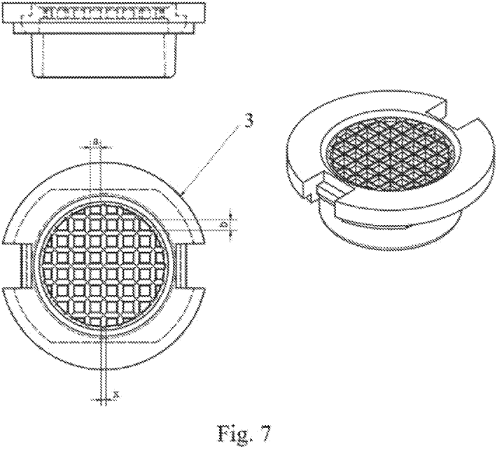

FIG. 6 shows a base of a mouthpiece of an inhaler according to the invention, in an isometric bottom view and an isometric top view, respectively;

FIGS. 7 - 9 show sieves in side, top and isometric views;

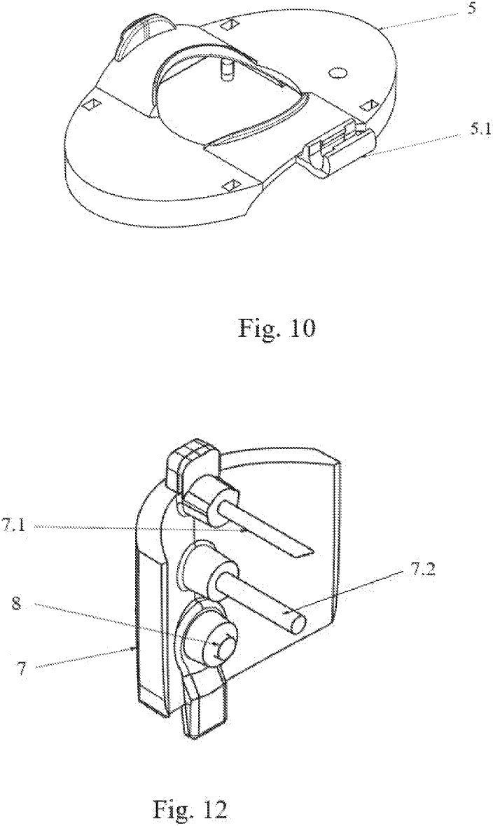

FIG. 10 shows a cover in an isometric top view;

FIG. 11 shows a capsule receiving element, in an isometric view presenting the bottom of the element, isometric view presenting the top of the element and in a top view of the element, respectively;

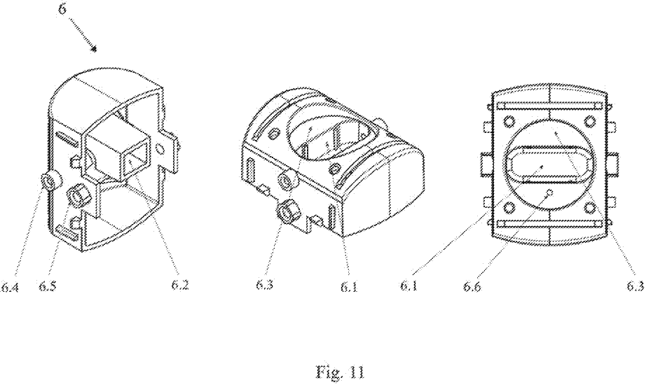

FIG. 12 shows a piercing mechanism of a press button of an inhaler according to the invention;

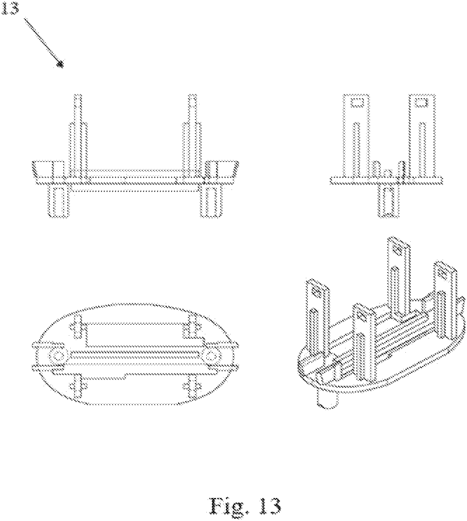

FIG. 13 shows a frame of an inhaler according to the invention;

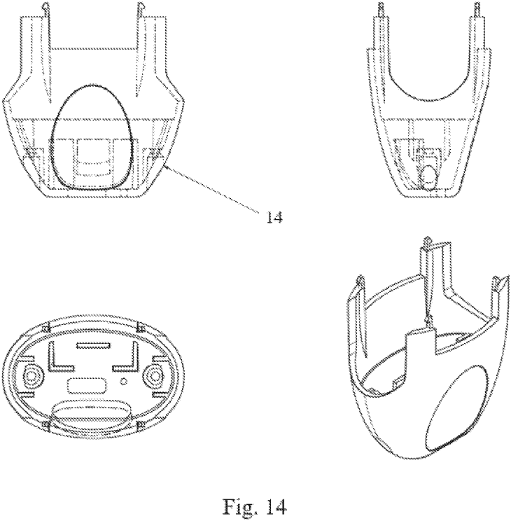

FIG. 14 shows a body of an inhaler according to the invention;

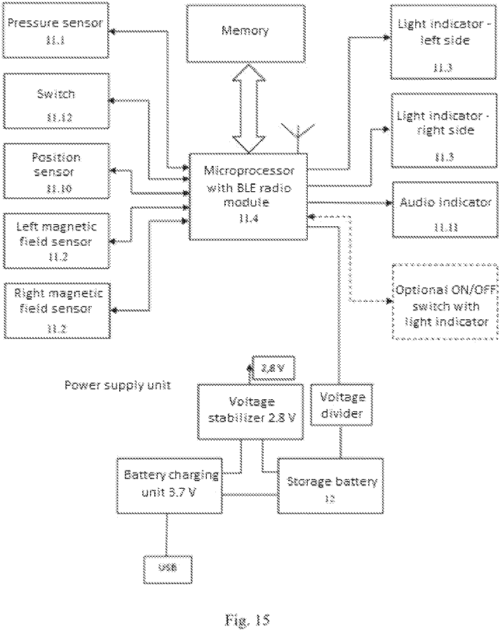

FIG. 15 shows a power supply diagram of an inhaler according to the invention;

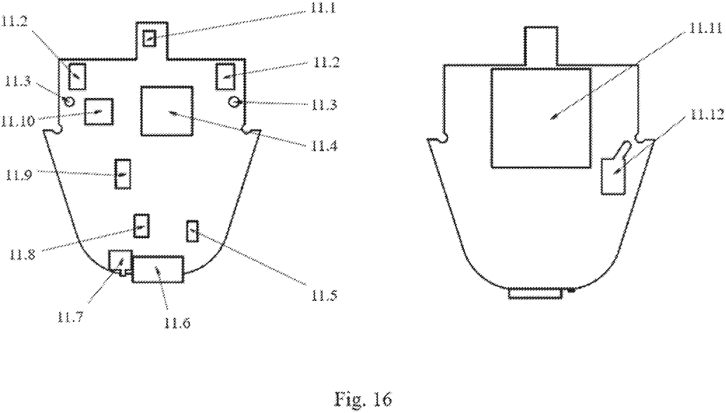

FIG. 16 shows an electronic board of an inhaler according to the invention;

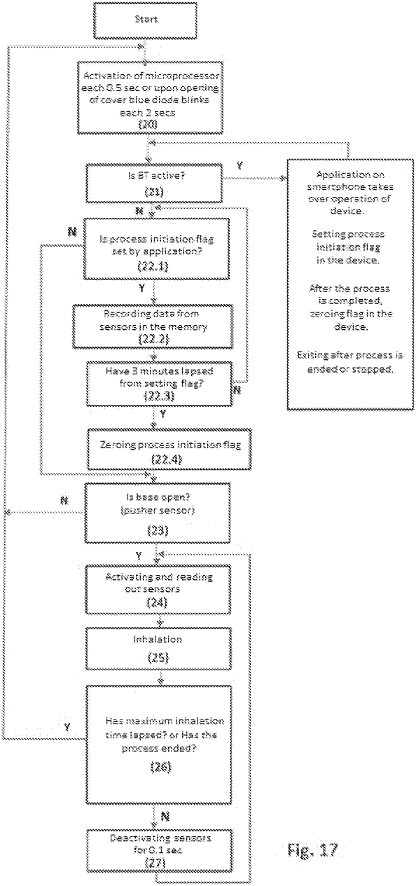

FIG. 17 shows a block diagram of an inhalation process according to a method of the invention;

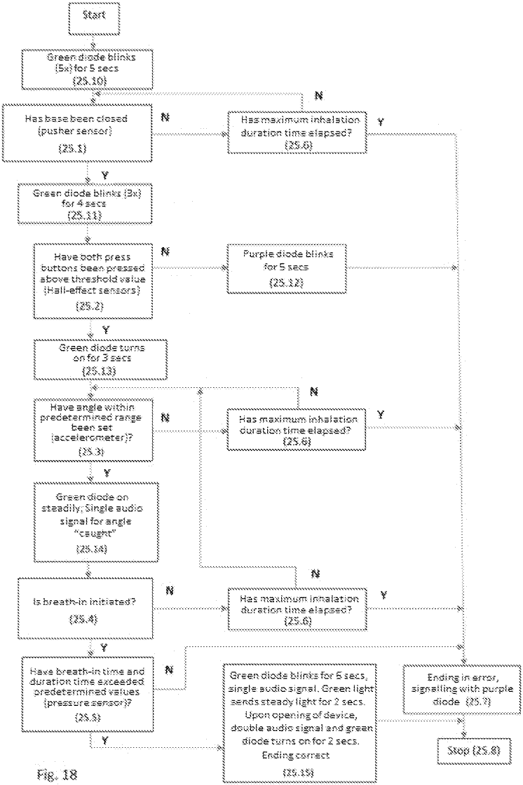

FIG. 18 shows a block diagram of operation in an inhalation step of an inhalation process according to the method of the invention.

In the embodiment shown in FIGS. 1 and 2, a capsule inhaler for administering a single dose of dry powder from a capsule comprises a body 14 coupled via catches with a cover 5 which is hingedly coupled to a base 4 of a mouthpiece 2. In an opening in the upper part of the base 4, a sieve 3 is mounted and the mouthpiece 2 that is closable from above by a housing 1. In the central “inner” part of the base 4 there is a rotatable chamber 4.2, to which, from two opposite sides, inlet channels 4.3 open, and through the channels air is drawn. On the base 4, a protrusion 4.1, for example, is provided to raise the base 4. To the base 4 the cover 5 with the opening is hingedly coupled, by means of a hinge.

The cover 5 covers a capsule receiving element 6 which for example may be made of transparent plastics to enable monitoring the inside of the element to determine whether a capsule got stuck, or whether it is properly inserted, or whether it has been pierced. The capsule receiving element 6 in the outer-central-upper part has a volume 6.3 along with the capsule chamber 6.1 into which the user locates a capsule and in this chamber 6.1 the capsule is pierced. Within the volume 6.3 the capsule gets drawn by the air flow from the chamber 6.1 onto the capsule and swirls within the rotatably chamber 4.2 provided in the base 4 of the mouthpiece 2, to release therapeutic substance. However, within the inner bottom part of the capsule receiving element 6, one of the long sides of the capsule chamber 6.1 adjoins a pressure drop chamber 6.2, communicating via an opening 6.6 with the volume 6.3 and into which a part of the electronic board 11 with a pressure sensor 11.1 is inserted from beneath.

The pressure sensor 11.1 enables the user to measure drug dosing intensity and time. Pressure measurement in a function of time makes it possible to determine duration time and force with which the user makes a breath-in during drug intake.

The capsule receiving element 6 on each of its two opposite walls has two openings - an opening 6.4 for a piercing element and below an opening 6.5 for a mandrel. Into the opening 6.4 for a piercing element, a piercing element 7.1, for example a needle, enters. The piercing element 7.1 is a part of the piercing mechanism and it is secured to a press button 7 over the mandrel 7.2. Into the opening 6.5 for a mandrel, a mandrel 7.2 enters, said mandrel being also secured on the press button 7. On the mandrel 7.2 a spring 9 is provided and the mandrel 7.2 as such stabilizes the motion of the press button 7 so that the press button 7 when pressed does not deviate from its longitudinal axis and the piercing element 7.1 always “enters” the same orientation via the opening 6.4 into the capsule chamber 6.1 and provides for reproducibility of piercing of the capsule, and thus reproducible effectiveness of drug release. Additionally, on the press button 7 below the mandrel 7.2 a magnet 8 is provided. The press buttons 7 are positioned at the height of the capsule receiving element 6.

The inhaler has two press buttons 7 coupled with the body 14 and cover 5 via upper and bottom catches. In the body 14 a frame 13 is located, releasably coupled with the body 14 by means of two connecting elements 15, for example screws or bolts. The frame 13, shown in FIG. 13, enables precise positioning of the pressure sensor 11.1 relative to a specially designed pressure drop chamber 6.2. The frame 13 provides stabilization and smooth movement of the press buttons 7 and joins all the constructive elements. In the frame 13 an electronic board 11 with a microprocessor 11.4 is positioned as well as a battery (or a storage cell) 12.

The electronic board 11 has at its sides two magnetic field sensors 11.2, for example a Hall-effect device, and light indicators 11.3, for example LEDs, positioned each in the proximity of the press buttons 7. Additionally, the magnetic field sensors 11.2 are positioned in the proximity of magnets so that they can react to the motion of the magnets 8 that move over the magnetic field sensors 11.2, and this makes it possible to record the velocity and depth at which each of the piercing elements 7.1 is pressed.

In addition, the electronic board 11 also comprises a switch 11.12, being a base opening sensor 4, coupled with the base 4 of the mouthpiece 2 via a coupling element 10, for example a pusher, that passes through an opening in the upper surface of the cover 5, which surface adjoins the base 4 of the mouthpiece 2. Activation of the inhaler is effected upon opening of the base 4 of the mouthpiece 2. The status of the pusher is changed mechanically by opening the base 4 of the mouthpiece 2 - detection of opening of the base 4 of the mouthpiece is recorded just thanks to the switch 11.12 and indicates initiation of the inhalation process.

In another embodiment, on the electronic board 11, coupled with the microprocessor 11.4, a position sensor 11.10 is arranged, such as for example 3-axial accelerometer that enables verification whether inhalation has been executed properly by verification whether the inhaler during inhalation was kept in a proper position, i.e. whether it had an inclination angle within the range 10°-20° +/-2° to the horizon, for example 15°.

In a further embodiment, on the electronic board 11, coupled with the microprocessor 11.4, an antenna 11.5 is arranged, for example a Bluetooth antenna, to enable wireless communication of the inhaler with a user device, such as for example a smartphone. The smartphone may have a suitable application installed therein and the principal functionalities of the application include:
educational module intended for working out proper habits during inhalation process;
inhalations history along with option for monitoring a single inhalation process and summary of the course of the process;
personalized list of administered drugs along with reminders;
reminder concerning the nearest planned inhalation;
map of pollen along with personalized warning alarms using geopositioning module;
weather widget temperature, air quality.

In one embodiment, on the electronic board 11, coupled with the microprocessor 11.4, a connector 11.6 is also positioned, for example a USB-C-type connector, to enable charging of the device powered by means of a battery 12 that gets discharged during operation.

In another embodiment, on the electronic board 11, coupled with a microprocessor 11.4, an on/off switch is arranged, for example positioned at the front of the housing and having a LED RGB light indicators. This switch enables manual turning on and/or off the inhaler when necessary, for example, to limit power consumption in non-use of the inhaler or when it is necessary to protect the device against unintended activation, for example when the device has been turned on inadvertently, for example in a bag.

In a further embodiment, in case of problems in operation of the inhaler, the settings of the device may be reset, and this is possible due to implementation, on the electronic board 11, of a resetting element 11.7, for example a resetting switch, connected to a microprocessor 11.4.

In another embodiment, on the electronic board 11, coupled with a microprocessor 11.4, a real time clock 11.8 is arranged, to enable during autonomous operation of the device without pairing to a mobile application, recording the precise time when measurement is taken.

In another embodiment, inhaler, on the electronic board 11, coupled with a microprocessor 11.4, has an audio indicator 11.11, that along with the light indicator 11.3 at each step of the inhalation process, when errors occur, transmits a corresponding alert to the user.

In a further embodiment, the inhaler comprises a replaceable sieve 3 to enable personalization of the inhaler to drugs to be administered to a specific user. For example, the sieve 3 shown in FIG. 7 may have a mesh with rectangular openings of a width a of 0.97 mm +/-0.03 mm and a height b of 1.00 mm +/-0.03 mm and a spacing X between the openings of 0.5 mm +/-0.03 mm.

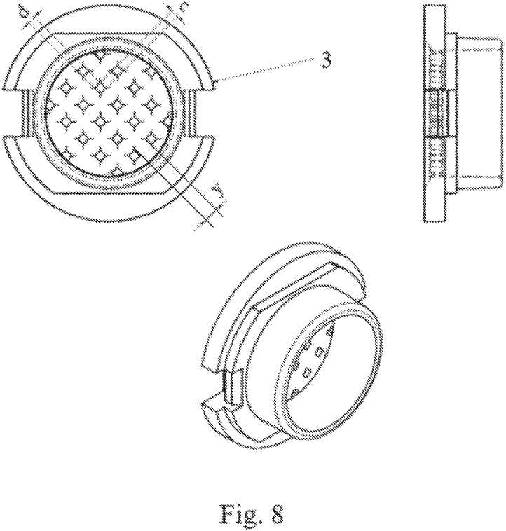

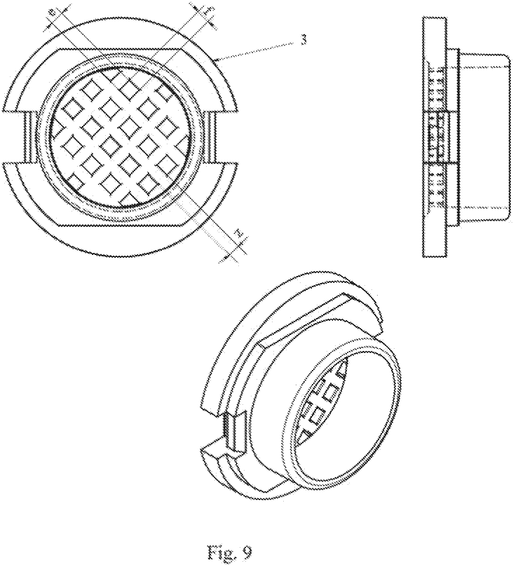

In another embodiment, the sieve 3 shown in FIG. 8 comprises a mesh with rectangular openings of a width c of 0.7 mm +/-0.02 mm and a height d of 1.1 mm +/-0.03 mm and a spacing Y between the openings of 1.4 mm +/-0.03 mm.

In a further embodiment, the sieve 3 shown in FIG. 9 comprises a mesh with rectangular openings of a width e of 1.11 mm +/-0.03 mm and a height ƒ of 1.1 mm +/-0.03 mm and a spacing Z between the openings of 0.93 mm+/-0.03 mm.

In another embodiment, the inhaler has a replaceable mouthpiece 2 with a decreased clearance of the inner diameter of the mouthpiece 2 as shown in FIG. 4a.

In a further embodiment, the inhaler has a replaceable mouthpiece 2 with a constant value of the clearance of the inner diameter of the mouthpiece 2, as shown in FIG. 4b.

In a further embodiment, the electronic board 11 of the inhaler shown in FIGS. 15 and 16 comprises a microprocessor with a BLE radio module, for example CC2650F128RGZR, connected to the following:
pressure sensor 11.1, for example BMP 280;
sensor 11.2 of opening of the base 4, for example SKRTLAE010;
position sensor 11.10, for example MPU6050;
left and right magnetic field sensor 11.2, for example 634-SI7210-B-00-IV and 634-SI7210-B-03-IV;
left and right light indicator 11.3, for example KPFA-3010RGBC-11;
audio indicator, for example SMT-1640-S-2-R;
optional on/off switch, for example with RGB LED signalling;
memory 11.9 such as EEPROM, for example AT24CM02, to enable the inhaler to record several complete operational cycles.

Additionally, in an embodiment, the microprocessor 11.4 has a power supply unit shown in FIG. 15, comprising: a voltage stabilizer 2.8 V (for example TC1015-2.8), LiPo storage battery 3.7 V minimum 380 mAh, voltage divider, storage battery charging unit 3.7 V (for example LTC4054) coupled with a connector 11.6, for example a USB-C socket.

All the embodiments related to a capsule inhaler according to the invention are also applicable to a monitoring method, a computer program and a computer program product according to the invention.

User who wishes to perform inhalation with the use of an inhaler according to the invention should prepare the device for use, first of all by checking whether the device is charged. If the device is not connected to a smartphone, a blue light blinks and this means that it is ready to be used. Before the first use, the user decides whether he/she wishes to use a specially designed application to monitor regularity of the inhalations performed. The application has also an educational module aimed at training the user in proper performing of the therapeutic process (inhalation).

If the user wishes to operate the inhaler with the use of the application, he/she has to download it first to his/her smartphone. The user installs the application in his/her smartphone. In order to pair the device with the application, the user accepts connection between the devices by means of a Bluetooth module. Upon installation of the application the user configures it according to the user’s guide.

The user with inhaler thus prepared, with the application activated and with a blister of capsules with drug, initiates inhalation process. The user holds the inhaler in one hand and with the other hand he/she grasps the housing 1 of the device or with his/her thumb levers the protrusion 4.1. (FIG. 6) positioned on the base 4 of the mouthpiece 2 to open the device. The module mouthpiece unit that comprises the mouthpiece 2 (FIGS. 4), sieve 3 (FIGS. 7-9) and base 4 of the mouthpiece 2 (FIGS. 5 and 6) and the housing 1 (FIG. 3) opens upwards and pivots rearwards from the device. This opening of the device causes that pressure on the coupling element 10 (FIG. 2) is released. The movement resulting from releasing of the coupling element 10 also releases pressure on the switch 11.12 (FIG. 16b). The switch 11.12 is a part of electronics that controls the inhaler. The switch 11.12 is positioned on the electronic board 11 (FIGS. 2 and 16), positioned inside the body 14 of the inhaler (FIG. 14). The electronic board 11 may be for example a PCB made in four-layer technology, with components at both sides thereof (FIG. 16).

Release by the coupling element 10 of the pressure on the switch 11.12 causes generating of a signal read by the software that controls the inhaler operation as “device open”. The signal transmitted by the electric element, when generating the information “device open”, causes release of a light signal by diodes 11.3 positioned on the same electronic board 11. The diodes 11.3, at the moment when the inhaler is activated, start blinking green. At the same time, due to the use of the Bluetooth module being also the antenna 11.5, the microprocessor 11.4 positioned on the electronic board 11 sends a signal to the user’s smartphone. In the application, for example on the smartphone screen, a message “Insert capsule and close the device” appears. At this moment the user removes a capsule from a blister and positions it in a special capsule chamber 6.1 arranged in a capsule receiving element 6 (FIG. 11). When this operation is completed, the user closes the inhaler. The module mouthpiece system comprising the mouthpiece 2 (FIGS. 4), sieve 3 (FIGS. 7-9) and the base 4 of the mouthpiece 2 (FIGS. 5 and 6) and the housing 1 (FIG. 3) is closed downward to pivot the front of the device. If after the device is open the user does not perform any operation, after about 3 minutes the device will return to a standby mode.

After the capsule is in place and the device is closed, the user grasps the devices so that he/she encloses the inhaler with his/her hand. The application sends a message: “When keeping the device vertically press and release side press buttons. Green light will indicate that the capsule has been pierced properly”. The back side of the inhaler defines a hinge 5.1, positioned on the cover 5 (FIG. 10). The back of the inhaler is enclosed by the user’s palm and thus the user may press simultaneously, using his/her index finger and thumb, the press buttons 7 (FIG. 12), positioned at both sides of the device. Before the press buttons 7 are pressed, the user may raise the device at the level of his/her eyes and state, looking through the capsule receiving element 6 (FIG. 11), whether in fact the capsule is positioned within the capsule chamber 6.1. If it is so, the user maintains the inhaler vertically and presses concurrently the press buttons 7. Needles 7.1 arranged on the press buttons 7, due to the slide movement of the press buttons 7 enabled by the use of springs 9 (FIG. 2), pierce the capsule. In the press buttons 7, magnets 8 are mounted (FIG. 12). The electronic board 11 has at its sides two Hall-effect sensors, “Hall-effect devices” 11.2 that react to the movement of the magnet 8. The signal transmitted by the Hall-effect devices 11.2 generates a numeric value of the signal. Trespassing of a threshold value causes the microprocessor installed or application to approve the process and this is indicated to the user by activation of steady green light. If for some reason a suitable signal value is not reached, the diodes 11.3 will turn purple and the application will show a message “Purple light indicates that the capsule has been pierced improperly. Replace it an initiate inhalation again.”.

If the user has pierced the capsule properly, the press buttons 7 are released to return to the initial position due to the use of the spring 9. The signal generated by the Hall-effect devices 11.2 caused by the movement of the magnets 8 has returned to the initial status and then the user receives the following information from the application: “Before inhalation remove the cap and make a shallow breath-in outside the inhaler area. Next put the inhaler mouthpiece into your mouth.”. The device is ready for further process and thus it sends steady green light. Upon removal of the housing 1 (FIG. 2), the user clicks “Next” in the application. The user receives a message “Find the proper angle for the inhaler. Remember to keep upright posture”.

The user makes a movement with the inhaler to put it into the vertical position. Setting of the proper angle is possible thanks to the accelerometer 11.10 positioned on the electronic board 11. A change in the angle of the device causes that by means of the accelerometer 11.10 controlled by the microprocessor 11.4 the position in the XYZ coordinate of the device is monitored. In the application a message appears “When keeping the proper posture take a deep breath-in” and in the application, for example at the sides of the screen, two arrow heads appear. When the device reaches the proper angle, the arrow heads in the screen merge into one line and change their colour into green, and the diodes 11.3 send a steady green light. Apart from the light indication, the device also generates a single audio signal. This is possible thanks to the use of the audio indicator 11.11 arranged on the electronic board 11. When the user hears the audio signal, he/she makes a breath-in, inserts the inhaler mouthpiece 2 into his/her mouth and clenches it. Before the user puts the mouthpiece 2 into the mouth, he/she has to release air to empty the lungs. Upon insertion of the mouthpiece 2 into the mouth he/she takes a strong, deep and possibly quick breath-in. The device, due to the use of the pressure sensor 11.1 arranged on the electronic board 11, may monitor pressure drop in the inhaler.

Pressure drop dP, correspondingly to the type of the inhaler used, effects the flow volume of the air Q caused by the user breathing-in. During the breath-in the pressure drop causes the air to flow. The air is drawn into the device by two inlet channels 4.3. The air reaches the rotatable chamber 4.2 arranged in the base 4 of the mouthpiece 2. The air generates a swirl and at the same time a negative pressure that draws the pierced capsule from the capsule chamber 6.1. The capsule makes a rotary motion and releases the drug through the openings resulting from piercing. The drug is composed of two kinds of particles: fine particles of the therapeutic substance and coarse particles of the carrier. These two particles are joined during the drug manufacturing process under mechanic forces and not chemical bonds. As a result of drawing the agglomerate from the capsule, as well as circulation of air, the agglomerate breaks into two fractions, a fine one and a coarse one. The capability of the user to draw the proper amount of air determines effectiveness of deagglomeration of the drug structure. As a result an aerosol is produced i.e. a mixture of air with therapeutic substance and carrier particles suspended therein. During breathing-in by the user, the aerosol reaches upper respiratory tract. Effective de-aggregation increases chance for the fine particle fraction comprising the therapeutic substance to reach the lower portion of the respiratory tract and become “absorbed” into the bronchioles. Obtaining of the proper air flow is a necessary condition for high probability of providing a proper therapeutic dose. With the use of a formula to determine relations between the pressure drop and the air flow, optimum ranges of pressure drop to be obtained are determined that are required to enable a considerable probability of providing proper therapeutic dose. The pressure sensor 11.1 positioned on the electronic board 11, during assembly of the device, is inserted into a specially designed pressure drop chamber 6.2, that adjoins the capsule chamber 6.1, and the pressure drop chamber 6.2 is communicated via an opening 6.6 with a volume 6.3 that is positioned along with the capsule chamber 6.1 in the upper part of the capsule receiving element 6.

The user breathes in. The device sends steady green light until a pressure change is stated. The diodes 11.3 blink green. At this time the user should hold his/her breath for 5 seconds. The diodes 11.3 blink until a minimum time of 5 seconds elapses. The application counts down 5 seconds. When the minimum of 5 seconds is reached, the device sends steady green light. The application shows a message: “Have you held your breath for 5 seconds?”.

The user breathes the air out. When the user confirms or denies holding his/her breath, the application generates a message “Have you removed the tablet?”. The user opens the inhaler similarly as at the beginning. Release of the coupling element 10 causes that a signal is generated to the device and to the application. The user cleans the inhaler and closes it. Upon confirming, the user receives an inhalation process report. When the user clicks “Next” he/she receives a message to confirm complete inhalation and to show the time for the next inhalation. When the button “End” is pressed, two audio signals are generated, and the inhaler buttons send steady green light. After 3 seconds the device passes into a standby phase.

In the embodiment shown in FIGS. 17 and 18, a method for monitoring and controlling a substance intake process is presented, with the use of an inhaler as described above.

When the inhaler is on, the method for monitoring intake of a substance with the use of a powder inhaler according to the invention controls the moment of activation of the inhaler from the standby phase by a step 20 of periodical activation of the processing unit, for example a microprocessor 11.4, each 0.5 sec when triggering each 2 seconds a blue light signal by the diode 11.3. When the user initiates the substance receiving procedure, the inhaler executes a step of verification 21 whether Bluetooth is active or not. If Bluetooth is active and the inhaler finds the user’s device to which it may communicate and which has an installed application enabling controlling the inhalation process, the application takes over control of the inhaler and at the beginning of the process it sets a flag “process initiated by the application”, i.e. initiation of the process, to be zeroed at the end of the inhalation process or after a step 26 of checking whether a maximum inhalation duration time elapsed or verifying whether inhalation step is completed. Setting of a flag is a signal for the inhaler that in the event of broken connection (e.g. discharge of the battery in the smartphone) the data from sensors are recorded 22.2, for example such sensors as a switch 11.12, a magnetic field sensor 11.2, a pressure sensor 11.1, a position sensor 11.10, into the inhaler memory, and then checking is executed (checking step 22.3) whether a predetermined duration time elapsed, for example 3 minutes from the moment of setting the process flag. Upon re-establishment of Bluetooth connection the application will retrieve the recorded data and record them in the inhalations history in the application.

If Bluetooth remains inactive and a predetermined duration time elapsed, the recording step 22.2 is stopped and the settings of the inhaler memory cell are reset 22.4 by zeroing of the flag set.

Steps of the inhalation process executed by the application are identical as when Bluetooth is not active. Upon completion or stopping of the inhalation process the application is exited and the operation with the application by the user is ended.

If Bluetooth is inactive, the steps are executed by the microprocessor 11.4 on the electronic board 11 and respective sensors, for example such as a switch 11.12, magnetic field sensor 11.2, pressure sensor 11.1, position sensor 11.10, real time clock 11.8.

Below an embodiment will be described when Bluetooth is inactive.

After the step of verification 21 of active state of Bluetooth, when Bluetooth is inactive, a step of verification of the process status in the inhaler memory is executed, consisting on a check 22.1 whether a flag “process initiated by the application” has been set. If the flag is set, the steps of recording 22.2 data from the sensors, checking 22.3 whether the predetermined duration time has elapsed, and resetting 22.4 the flag are executed as described above. If the flag is not set, the method passes to the step of verifying 23 opening of the base 4. The step of verification 23 of opening of the base 4 of the mouthpiece 2, checks opening by monitoring the coupling element 10, for example a pusher. Opening of the base 4 of the mouthpiece 2 causes release of pressure on the coupling element 10 and this in turn causes release of pressure on the switch 11.12 positioned on the electronic board 11 and connected to the microprocessor 11.4 and passing to a step of activating and reading 24 of the status of the sensors, for example such as a switch 11.12, magnetic field sensor 11.2, pressure sensor 11.1, position sensor 11.10, real time clock 11.8. Activation 24 consists on reading out the statuses of the sensors adopted as reference for further monitoring of changes.

Absence of action of opening of the base 4 of the mouthpiece 2 cases that the method returns to the step 20 of periodic activation.

If in the step of verification 23 opening of the base 4 of the mouthpiece 2 is detected, and the step of constant activation and reading out 24 the statuses of the sensors is executed, the method passes to the inhalation step 25. The diodes 11.3 start blinking 25.10 green intermittently for 5 seconds, to signalize to the user of the inhaler that the device is open and a capsule may be inserted, and then verification 25.1 of closing of the base 4 is executed via monitoring the coupling element 10. Closing of the base 4 causes again pressure of the coupling element 10 acting on the switch 11.12, and this makes it possible for the microprocessor 11.4 to recognize that the base is closed.

Activation as used herein should be understood as an activation after which the sensors are not set into a standby mode and the inhalation step 25 is executed which may be ended in the step 27 of setting the above indicated sensors into a standby mode, as specified below.

If the base 4 of the mouthpiece 2 is not closed, checking 25.6 is executed whether a maximum inhalation duration time has elapsed, for example a duration time of a range of 3 minutes, and after elapse of such duration time the inhalation step 25 ends in error 25.7.

If the base 4 is closed, the diodes 11.3 blink green thrice 25.11 intermittently for 4 seconds, and then the user presses concurrently two press buttons 7 and thus causes that two piercing elements 7.1 slidingly cause piercing of the capsule and this is related to the movement of magnets 8, and more specifically passing of the magnets 8 over magnetic field sensors 11.2. Pressing of the press buttons 7 is verified 25.2 by checking whether the signal from the magnetic field sensors 11.2, for example Hall-effect sensors (Hall-effect devices), exceed a predetermined threshold value that guarantees that the capsule is pierced.

If the signal from the magnetic field sensors 11.2 does not exceed the predetermined threshold value this means that the capsule has not been pierced properly and the diodes 11.3 start blinking purple 25.12 for 5 seconds and then the inhalation step 25 ends in error 25.7.

If the signal from the magnetic field sensors 11.2 exceeds the predetermined threshold value for the step, the diodes 11.3 start sending 25.13 green light steadily for 3 seconds and then via the position sensor 11.10, such as for example an accelerometer, position of the inhaler is verified 25.3.

If the inclination angle of the inhaler is not within the range of 10°-20° to the horizon, with a possible deviation from the vertical axis by +/-2°, checking 25.6 is executed whether a maximum inhalation duration time has elapsed, i.e. 180 seconds, and after elapse of the time the inhalation step 25 ends in error 25.7.

If the inhaler position is the desired position, the diodes 11.3 start sending green light steadily and a single audio signal is generated 25.14 by means of an audio indicator 11.12. The diodes go out each time if the inclination angle of the inhaler exceed the range of 10°-20° to the horizon with a possible deviation from the vertical axis by +/-2°. When the signal is audible the user may put the mouthpiece 2 into his/her mouth and breath-in.

Then, checking is executed whether breathing-in has been initiated by checking 25.4 the status of the pressure sensor 11.1 that monitors breath.

If the above checking 25.4 of the pressure sensor status shows absence of pressure drop dP, checking 25.6 is executed whether a maximum inhalation duration time has expired, i.e. 180 seconds, and upon elapse of this duration time the inhalation step 25 ends in error 25.7.

If checking 25.4 of the pressure sensor status shows a pressure drop dP, checking is executed whether the breath-in has been sufficiently strong and whether maintaining of the breath-in was sufficiently long, by verifying 25.5 the value of the breath-in force and the breath-in duration time. For example, it is checked whether maintaining the breath-in after it was initiated lasted for minimum of 5 seconds for proper intake of the substance by the user. The desired pressure drop may be calculated based on the dimensions and resistance of a given inhaler. For example, for an inhaler with a mouthpiece 2, with an internal opening ratio of the mouthpiece 2 being 5.20 mm (+/-0.15 mm), with an inlet channel 4.3 of a width of 4.22 mm (+/-0.03 mm) and a sieve 3 having dimensions a = 0.97 mm (+/-0.03 mm); b = 0.97 mm (+/-0.03 mm); X = 0.5 mm (+/-0.03 mm), minimum desired pressure drop dP being 14 hPA for the flow of 60 L/minute or 32 hPA for the flow of 90 L/minute.

If the duration time or the value of the breath-in force in step 25.5 do not exceed the predetermined values. The inhalation step 25 ends in error 25.7.

If the duration time or the value of the breath-in force in step 25.5 exceed the predetermined values, the diodes 11.3 start blinking 25.15 green intermittently for 6 seconds, and for another 2 seconds green steadily, and a single audio signal is generated by means of the audio indicator 11.11.

In one embodiment, after the step 25.5 of verification whether the duration time and the value of the breath-in force exceed the predetermined value also a check is executed whether the user opened the base 4 of the mouthpiece 2 and removed the used capsule from the chamber 6.1. If the sensor 11.12 detects that the base 4 of the mouthpiece 2 is open, the diodes 11.3 send green light for 2 seconds and a double audio signal is generated by means of the audio generator 11.11, and afterwards the inhalation step 25 ends 25.8.

In one embodiment not shown in the diagram, where the switch 11.12 does not detect opening and closing of the base 4 of the mouthpiece 2, a check 25.6 is executed whether a maximum inhalation duration time elapsed, i.e. 180 seconds, and upon elapse of such duration time the inhalation step 25 is ended 25.8.

When the inhalation step 25 is ended, a check 26 is executed whether the maximum inhalation duration time elapsed, i.e. the duration time exceeded 180 seconds or it is verified whether the inhalation step has ended.

If the inhalation step has ended or the maximum inhalation duration time elapsed, the method returns to the step of periodical activation 20 of the processing unit.

If the inhalation step has not ended or the maximum inhalation duration time has not elapsed, the above mentioned sensors are set 27 to a standby mode for 0.1 sec, and then the method passes to the step 24 of activating and reading out of the status of the sensors.

In an embodiment of an inhaler according to the invention where on the electronic board 11, coupled with a microprocessor 11.4, an on/off switch is positioned, the method of the invention does not include the step 20 of activating the microcontroller and it begins with the step 21 of verification whether Bluetooth is active.

Below a further embodiment will be described for a case where in the step of verification 21 is it detected that Bluetooth is active. In this case the monitoring method steps are substantially the same as when Bluetooth is not active, and the only difference is constituted by additional messages and light indicators that appear on the user device, for example a smartphone, as presented below.

Upon detection of active Bluetooth connection operation of the inhaler is taken over by the application on the smartphone. Blue light indication on the inhaler press button goes out. Upon activation in the application of the function/press button “Initiate inhalation” in the application of the smartphone a message appears: “Open the inhaler and the press buttons start blinking green”. After the step of verification 23 of opening of the base 4 of the mouthpiece 2, apart from the steps described above, if it is detected that the base 4 of the mouthpiece 2 is open, additionally in the application of the smartphone a request appears “Insert capsule and close the device”.

After the step of verification 25.1 of closing of the base 4 and detection that the base 4 is closed, in the application of the smartphone, a message confirming readiness of the device for piercing the capsule appears: “When keeping the device vertically, press and release side press buttons. Green light will indicate that the capsule has been pierced properly”.

After the step of verifying 25.2 whether the two press buttons 7 have been pressed, if a predetermined threshold value for the magnetic field sensors 11.2 is not exceeded, the diodes 11.3 turn purple, and in the application a message appears “Purple light indicates that the capsule has been pierced improperly. Replace it and initiate inhalation again.”. The user should then replace the improperly pierced capsule with a new one by repetition of the method steps beginning from verification 25.1 of closing of the base 4 and proceed further according to the following steps.

After the step of verifying 25.2 whether the two press buttons have been pressed, if the predetermined threshold value for the magnetic field sensors 11.2 is exceeded and the capsule has been properly pierced, a green diode will send steady light (25.13), and the application will show information on preparation for administering a drug dose: “Before inhalation take the caps off and make a light breath-in outside the area of the inhaler. Then put the inhaler mouthpiece into your mouth”. When,,Next″ in the application is clicked by the user, a message appears “Find the proper angle for the inhaler. Remember to keep the upright posture”. The user moves the inhaler to set it into a vertical position and the step of verification 25.3 of the inhaler position to the horizon begins, during which, for example, two arrow heads appear at the sides of the screen and when the inhaler reaches the proper angle, they merge into one line and change their colour into green.

If the proper angle is detected during verification 25.3 of the inhaler position, the step 25.14 is executed in which the diodes 11.3 start sending green light intermittently for 5 seconds and a single audio signal is generated by means of the audio indicator 11.12, and the application shows a message “When keeping the proper position take a deep breath-in”.

In a variant of the embodiment with Bluetooth active during the step of checking 25.4 of the status of the pressure sensor and verifying 25.5 the value of the breath-in force and the breath-in duration time, the application shows a timer providing the proper inhalation duration time and thereafter, if the duration time and pressure exceed predetermined values, the step 25.15 of communicating the user is executed.

After the step 25.15 of communicating the user, by means of light and audio signals, in the application a message containing request for confirmation of holding a breath for 5 seconds appears. Thereafter, the step 25.8 of ending inhalation 25 is executed and the application shows a summary of the inhalation process, after which the step 26 of checking whether the maximum inhalation duration time elapsed or verifying whether the inhalation step has ended.

If any of the operations within the inhalation step is not executed, i.e. when opening of the base is not detected, pressing of the press buttons is not detected, angle is not detected, pressure drop is absent, the device waits for 180 secs for the user’s reaction, i.e. enables the user to insert a capsule, to press the press buttons and to perform further operations related to inhalation. If within this time the user performs the respective operation, the method passes to a subsequent step. If the user fails to perform such operation, the device passes to a standby mode, i.e. the user will have to initiate the process again.

It should be kept in mind that the above described messages are provided as examples and they do not limit the specific wording of the messages. The messages in the application may be presented or recorded in any other manner to present the required contents.

In another embodiment, all the steps of the above described monitoring method may be executed by a computer program for monitoring intake of a substance, comprising instructions for execution of these steps.

In a further embodiment, all the steps of the above described monitoring method may be executed by a computer program product comprising a computer readable code.

All the embodiments related to the monitoring method according to the invention also apply to the capsule inhaler according to the invention.

As used herein, all terms “upper”, “lower” or the like throughout this text are provided as an example and do not limit the positioning of the inhaler elements or configuration thereof.

The embodiments shown herein are solely non-limiting indications related to the invention and cannot in any way limit the scope of protection defined by the patent claims. It should be understood that each technical solutions used in the inhaler according to the invention may be implemented by means of equivalent technologies without exceeding the scope of protection.

1. A capsule inhaler for administering a single dose of dry powder from a capsule, said inhaler comprising:
a body,
a mouthpiece having a sieve, a base, and a cover comprising an opening, the mouthpiece being coupled with the body,
a capsule receiver,
at least one press button with a capsule piercing mechanism comprising at least one piercing element and at least one magnet,
at least one electronic board, the electronic board comprising a microprocessor, at least one position sensor, at least one magnetic field sensor, at least one pressure sensor, and at least one light indicator, positioned in the proximity of the at least one press button,
a coupler coupling the electronic board with the base of the mouthpiece.

2. The inhaler according to claim 1, wherein the capsule piercing mechanism further comprises at least one spring.

3-5. (canceled)

6. The inhaler according to claim 1, wherein the electronic board comprises at least one of an audio indicator, a switch that, via the coupler, is coupled with the base, and an antenna, a connector, an on/off switch, a resetting element, and a real time clock, that are coupled with a microprocessor, and wherein a battery is arranged in a frame in proximity to the electronic board.

7-17. (canceled)

18. The inhaler according to claim 1, wherein the sieve comprises a mesh with rectangular openings of a width (a) 0.94 and 1 mm and a height (b) comprised between 0.97 and 1.03 mm and a spacing (X) between the openings of between 0.47 and 0.53 mm.

19. The inhaler according to claim 1, wherein the sieve comprises a mesh with rectangular openings of a width (c) comprised between 0.68 and 0.72 mm and a height (d) comprised between 1.07 and 1.13 mm and a spacing (Y) between the openings of between 1.37 and 1.43 mm.

20. The inhaler according to claim 1, wherein the sieve comprises a mesh with rectangular openings of a width (e) comprised between 1.08 and 1.14 and a height (f) comprised between 1.07 and 1.13 mm and a spacing (Z) between the openings of between 0.90 and 0.96 mm.

21. A method for monitoring intake of a substance using a capsule inhaler, the method comprising the following steps executed by a processing unit:
periodically activating the processing unit;
verifying whether Bluetooth is active;
verifying the status of the process in an inhaler memory;
verifying opening of a base of a mouthpiece by monitoring a coupling element;
activating and reading out of sensors;
verifying closing of the base of the mouthpiece by monitoring the coupling element;
verifying pressing of press buttons, by checking whether the signal from magnetic field sensors exceeds a predetermined threshold value;
verifying, by a position sensor, the inhaler position;
verifying the status of the pressure sensor that monitors breath-in,
verifying the value of the breath-in force and breath-in duration time;
ending the inhalation process; and
verifying ending of the inhalation process.

22. The method according to claim 21, wherein verifying the process status in the inhaler memory comprises verifying of the inhaler memory setting and, with a positive verification result of the inhaler memory setting, recording in the inhaler memory of the data from the sensors, verifying the counted-down duration time of the inhaler memory setting and zeroing of the inhaler memory cell setting, and then passing to the step of verifying opening of the base, and with a negative result of verifying the inhaler memory setting, passing to the step of verifying opening of the base of the mouthpiece.

23. (canceled)

24. The method according to claim 21, wherein verifying the process status the inhaler memory comprises verifying the inhaler memory setting and, with positive result of verifying the inhaler memory setting, recording in the inhaler memory of the data from the sensors and further verifying of the counted-down duration time of the inhaler memory setting, and with negative result of verification of the counted-down duration time of the inhaler memory setting, returning to the step of verifying the inhaler memory setting.

25. The method according to claim 24, wherein upon negative result of verifying opening of the base of the mouthpiece, the method returns to the step of periodic activating of the processing unit.

26. The method according claim 24, wherein upon positive result of verifying the breath-in force and breath-in duration time, additionally opening of the base of the mouthpiece is verified.

27. The method according to claim 21, further comprising in the step of checking whether the maximum inhalation duration time has elapsed, verifying the status of the inhalation step.

28. The method according to claim 21, wherein the step of verifying ending of the inhalation process is executed when at least one of the following events occurs: elapse of the maximum inhalation duration time, ending of the inhalation step, the method returns to the step of periodic activation of the processing unit.

29. The method according to claim 21, wherein the step of verifying ending of the inhalation process when at least one of the following events occurs: negative result of verifying lapse of the maximum inhalation duration time, ending of the inhalation step, a step of setting the sensors into a standby mode is executed.

30. The method according to claim 21, wherein following the step of setting the sensors into a standby mode, the method returns to the step of activating and reading out the status of the sensors.

31. (canceled)

32. The method according to claim 1, wherein after each negative result of verification of closing of the base, verification of the inhaler position and with negative result of a check of the pressure sensor status, a step of verifying whether the duration time exceed a predetermined value is executed.

33-34. (canceled)

35. The method according claim 21, wherein in the step of activating and reading out the status of the sensors, the status of at least one of the following: switch, magnetic field sensor, pressure sensor, position sensor, real time clock is read out.

36-38. (canceled)

39. The method according to claim 21, wherein the step of verifying the inhaler position is effective when the inclination angle of the device is within a range of 10°- 0° +1-2° to the horizon.

40. The method according to claim 21, wherein with positive result of the step of verifying Bluetooth activity, the method is executed by an application on a user device.

41. (canceled)

42. A computer program embodied on a non-transitory computer readable storage medium for monitoring intake of a substance, comprising instructions for executing the method defined in claim 21.

43. A computer program product embodied on a non-transitory computer readable storage medium, comprising a computer readable code, executing steps of the method defined in claim 21.
